(19) United States
(12) Patent Application Publication
(10) Pub. No.: US 2023/0082564 A1
(43) Pub. Date: Mar. 16, 2023

(54) COUPLED PHYSIOLOGICAL SIGNAL MEASUREMENT METHOD, COUPLED PHYSIOLOGICAL SIGNAL MEASUREMENT SYSTEM AND GRAPHIC USER INTERFACE

(71) Applicant: INDUSTRIAL TECHNOLOGY RESEARCH INSTITUTE, Hsinchu (TW)

(72) Inventors: Heng-Yin CHEN, Zhubei City (TW); Yun-Yi HUANG, Pingtung City (TW); Min-Hsuan LEE, Ji'an Township (TW); Yu-Chiao TSAI, Tainan City (TW)

(73) Assignee: INDUSTRIAL TECHNOLOGY RESEARCH INSTITUTE, Hsinchu (TW)

(21) Appl. No.: 17/586,514

(22) Filed: Jan. 27, 2022

(30) Foreign Application Priority Data
Sep. 10, 2021 (TW) 110133792

(51) Int. Cl. A61B 5/0533; A61B 5/389; A61B 5/00

(52) U.S. Cl. CPC A61B 5/0533; A61B 5/389; A61B 5/7257; A61B 5/743; A61B 5/7225

(57) ABSTRACT

A coupled physiological signal measurement method, a coupled physiological signal measurement system and a graphic user interface are provided. The coupled physiological signal measurement method includes the following steps. An original myoelectric signal is captured. A capacitance value of a skin is obtained. The original myoelectric signal is compensated according to the capacitance value of the skin. The step of compensating the original myoelectric signal according to the capacitance value includes the following steps. The original myoelectric signal is decomposed to obtain several myoelectric sub-signals corresponding to several frequencies, wherein each myoelectric sub-signal has an amplitude variation. The amplitude variations of the myoelectric sub-signals are respectively adjusted according to the capacitance value of the skin. The adjusted myoelectric sub-signals are merged to obtain a compensated myoelectric signal.

This application claims the benefit of Taiwan application Serial No. 110133792, filed Sep. 10, 2021, the disclosure of which is incorporated by reference herein in its entirety.

### TECHNICAL FIELD

The disclosure relates to a signal measurement method, a signal measurement system and a graphic user interface, and also relates to a coupled physiological signal measurement method, a coupled physiological signal measurement system and a graphic user interface.

### BACKGROUND

As people are placing more and more focus on the requirements of health management, various physiological signal sensing devices are provided one after another. Of the devices for sensing physiological signal, resistive physiological signal sensing devices are most common. For example, the resistive physiological signal sensing devices are used in various fields, such as sports fitness, health care, and long-term care.

However, to obtain satisfactory signal quality, conventional resistive physiological signal sensing devices need to have close contact with the skin. Once the conventional resistive physiological signal sensing devices no longer maintain close contact with the skin, meaningful measurement signals cannot be obtained. On the other hand, the practice of adhering conventional resistive physiological signal sensing devices on the skin using an adhesive always generates side effects such as redness and irritation. Therefore, it has become a prominent task for the industries to provide a coupled physiological signal measurement system capable of obtaining satisfactory measurement signal without having to maintain close contact with the skin.

### SUMMARY

According to one embodiment, a coupled physiological signal measurement method is provided. The coupled physiological signal measurement method includes the following steps. An original myoelectric signal is captured. A capacitance value of a skin is obtained. The original myoelectric signal is compensated according to the capacitance value of the skin. The step of compensating the original myoelectric signal according to the capacitance value includes the following steps. The original myoelectric signal is decomposed to obtain several myoelectric sub-signals corresponding to several frequencies, wherein each of the myoelectric sub-signals has an amplitude variation. The amplitude variations of the myoelectric sub-signals are respectively adjusted according to the capacitance value of the skin. The adjusted myoelectric sub-signals are merged to obtain a compensated myoelectric signal.

According to another embodiment, a coupled physiological signal measurement system is provided. The coupled physiological signal measurement system includes a myoelectric signal sensing unit, a skin sensing unit and a compensation unit. The myoelectric signal sensing unit is configured to capture an original myoelectric signal. The skin sensing unit is configured to obtain a capacitance value of a skin. The compensation unit is configured to compensate the original myoelectric signal according to the capacitance value of the skin. The compensation unit includes a decomposer, an adjuster and a merger. The decomposer is configured to decompose the original myoelectric signal to obtain several myoelectric sub-signals corresponding to several frequencies, wherein each of the myoelectric sub-signals has an amplitude variation. The adjuster is configured to adjust the amplitude variations of the myoelectric sub-signals respectively according to the capacitance value of the skin. The merger is configured to merge the adjusted myoelectric sub-signals to obtain a compensated myoelectric signal.

According to an alternate embodiment, a graphic user interface is provided. The graphic user interface includes a first wave window, a skin sensing information window and a second wave window. The first wave window is configured to display an original myoelectric signal. The skin sensing information window is configured to display a capacitance value of a skin. The second wave window is configured to display a compensated myoelectric signal. The original myoelectric signal is adjusted according to the capacitance value of the skin to obtain the compensated myoelectric signal.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1 is a schematic diagram of a coupled physiological signal measurement system according to an embodiment.

FIG. 2 is a block diagram of a coupled physiological signal measurement system according to an embodiment.

FIG. 3 is a flowchart of a coupled physiological signal measurement method according to an embodiment.

FIG. 4 is a schematic diagram illustrating an original myoelectric signal and an ideal myoelectric signal.

FIG. 5 is a schematic diagram of adjusting a particular myoelectric sub-signal.

FIG. 6 is a schematic diagram of a skin sensing unit according to an embodiment.

FIG. 7 is a block diagram of a coupled physiological signal measurement system according to an embodiment.

FIGS. 8A to 8B are flowcharts of a coupled physiological signal measurement method according to an embodiment.

FIG. 9 is a schematic diagram illustrating the voltage sensing signal.

FIG. 10 is a schematic diagram illustrating how the reference time constant is obtained using a differential algorithm.

FIG. 11 is a schematic diagram of a skin sensing unit according to another embodiment.

FIG. 12 is a block diagram of a coupled physiological signal measurement system according to an embodiment.

FIG. 13 is a flowchart of a coupled physiological signal measurement method according to an embodiment.

FIG. 14 is a schematic diagram illustrating various capacitive impedance curves.

FIG. 15 is a block diagram of a coupled physiological signal measurement system according to an embodiment.

FIG. 16 is a flowchart of a coupled physiological signal measurement method according to an embodiment.

FIG. 17 is a block diagram of a coupled physiological signal measurement system according to an embodiment.

FIG. 18 is a flowchart of a coupled physiological signal measurement method according to an embodiment.

FIG. 19 is a schematic diagram of a graphic user interface according to an embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

The embodiments of the present disclosure is directed to a coupled physiological signal measurement method, a coupled physiological signal measurement system and a graphic user interface. After an original myoelectric signal is obtained, the original myoelectric signal can be compensated according to the capacitance value of the skin to obtain a compensated myoelectric signal. The compensated myoelectric signal resolves the problem of impedance mismatch, such that the coupled physiological signal measurement system adopting low pressure sensing or non-contact sensing also can obtain a measurement result with high accuracy.

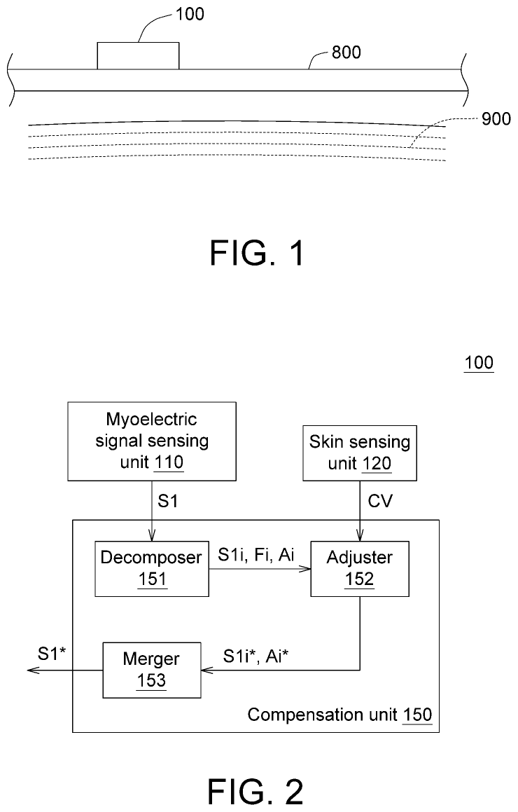

Referring to FIG. 1, a schematic diagram of a coupled physiological signal measurement system 100 according to an embodiment is shown. In the present embodiment, the coupled physiological signal measurement system 100 does not need to have close contact with the skin 900. The coupled physiological signal measurement system 100 can be disposed on an inner side of the fabric 800. For example, the coupled physiological signal measurement system 100 can be placed in an inner side opening of a functional clothing/pant, such that the coupled physiological signal measurement system 100 can be lightly attached to the skin 900 with low pressure. Or, the coupled physiological signal measurement system 100 can be placed in a strap sandwich of a backpack and has an indirect contact with the skin 900 through a layer of nylon cloth.

The said low pressure sensing or non-contact sensing needs to overcome the situation that the capacitance value of the skin 900 being susceptible to interference and the measurement being inaccurate as the capacitance value of the skin 900 increases. These problems occur mainly due to the impedance mismatch between the electrode and the skin 900. For example, physical contact and perspiration of the skin 900 cause the capacitance value to change; signals may drift when the contact surface between the electrode and the skin 900 moves.

In an embodiment of the present disclosure, the drifting or fading of signals can be compensated through dynamic compensation. The capacitance value between the electrode and the skin can be detected through various designs. Signal variations can be compensated using an algorithm according to the capacitance value of the skin.

Referring to FIG. 2, a block diagram of a coupled physiological signal measurement system 100 according to an embodiment is shown. The coupled physiological signal measurement system 100 includes a myoelectric signal sensing unit 110, a skin sensing unit 120 and a compensation unit 150.

The myoelectric signal sensing unit 110 is configured to capture an original myoelectric signal S1. The myoelectric signal sensing unit 110 can be formed of electrode sheet, chip, and circuit board. The original myoelectric signal S1 can be an electromyography (EMG) signal, an electrocardiogram (ECG) signal or an electroencephalography (EEG) signal. The original myoelectric signal S1 may have been seriously interfered and lose its accuracy.

The skin sensing unit 120 is configured to obtain a capacitance value CV of a skin 900 (illustrated in FIG. 1). The skin sensing unit 120 can be formed of resistor, capacitor, and chip. The capacitance value CV of the skin 900 can accurately reflect impedance mismatch.

The compensation unit 150 is configured to compensate the original myoelectric signal S1 to increase measurement accuracy according to the capacitance value CV of the skin 900. The compensation unit 150 can be formed of circuit, chip, and circuit board.

The compensation unit 150 includes a decomposer 151, an adjuster 152 and a merger 153. Functions of each element are disclosed below. The compensation unit 150 decomposes the signal using the decomposer 151, adjusts the decomposed signals using the adjuster 152, and merges the adjusted signals using the merger 153 to obtain final compensation results. Details of the operations of each of the elements disclosed above are disclosed below with a flowchart.

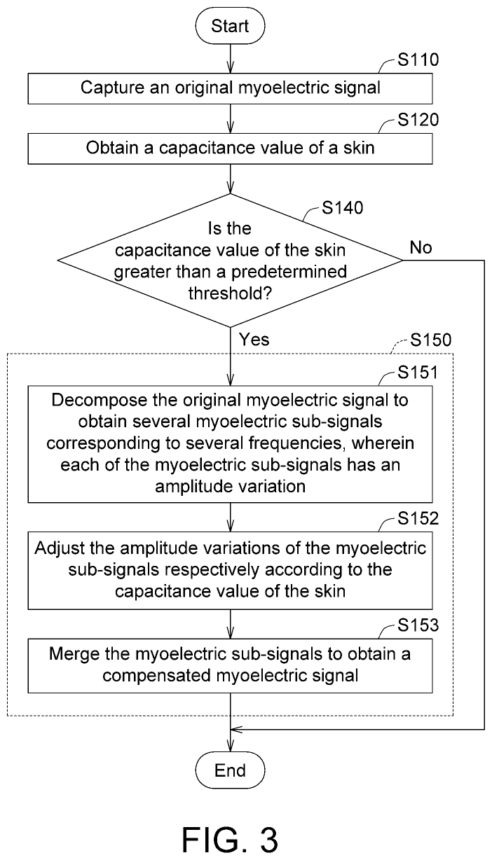

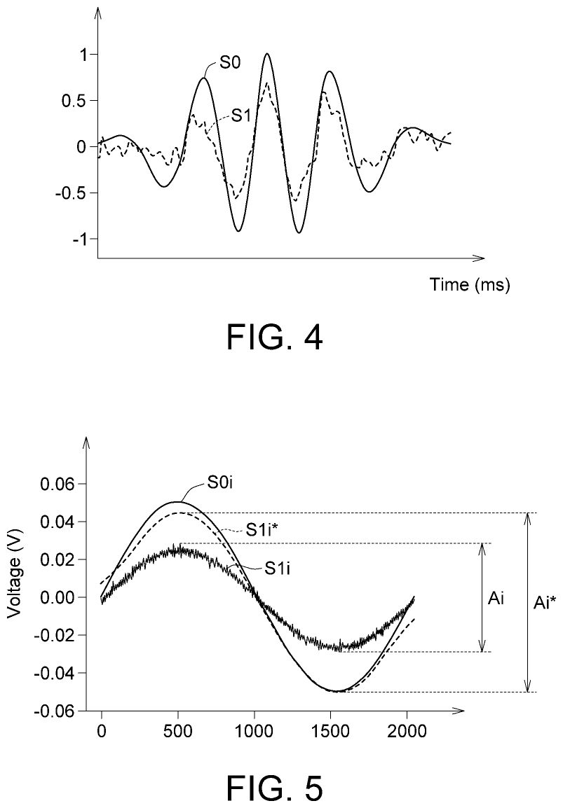

Referring to FIG. 3, a flowchart of a coupled physiological signal measurement method according to an embodiment is shown. In step S110, an original myoelectric signal S1 is captured by the myoelectric signal sensing unit 110. The myoelectric signal sensing unit 110 performs low pressure sensing or non-contact sensing on the skin 900 to obtain the original myoelectric signal S1. Since the myoelectric signal sensing unit 110 does not tightly press the skin 900, the original myoelectric signal S1 is susceptible to interference. Referring to FIG. 4, a schematic diagram illustrating an original myoelectric signal S1 and an ideal myoelectric signal S0 is shown. As indicated in FIG. 4, the ideal myoelectric signal S0 is free of interference and has larger amplitudes and simpler frequencies. The interfered original myoelectric signal S1 has smaller amplitudes and is interfered in various frequencies.

Then, the method proceeds to step S120, a capacitance value CV of a skin 900 is obtained by the skin sensing unit 120.

Afterwards, the method proceeds to step S140, whether the capacitance value CV of the skin 900 is greater than a predetermined threshold is determined by the compensation unit 140. If the capacitance value CV is greater than the predetermined threshold, the method proceeds to step S150; if the capacitance value CV is not greater than the predetermined threshold, the method terminates. The predetermined threshold is set according to the user's age, weight, gender, height or medical history. Following compensation is activated only when the capacitance value CV is greater than the predetermined threshold.

In step S150, the original myoelectric signal S1 is compensated by the compensation unit 150 according to the capacitance value CV of the skin 900. Step S150 includes steps S151 to S153.

In step S151, the original myoelectric signal S1 is decomposed by the decomposer 151 to obtain several myoelectric sub-signals S1i corresponding to several frequencies Fi, wherein each myoelectric sub-signal S1i has an amplitude variation Ai. The amplitude variation Ai can be a difference between an amplitude crest and an amplitude trough, or the difference between the center point of the AC wave signal and the amplitude crest or amplitude trough. As indicated in Table 1, the original myoelectric signal S1 can be decomposed into several myoelectric sub-signals S1i “S11, S12, . . . , S1n” whose frequencies Fi respectively are “F1, F2, . . . , Fn”, and the amplitude variations Ai corresponding to the frequencies Fi respectively are “A1, A2, . . . , An”.

TABLE 1

| Myoelectric sub-signal S1i | Frequency Fi | Amplitude variation Ai |
|---|---|---|
| S11 | F1 | A1 |
| S12 | F2 | A2 |
| . . . | . . . | . . . |
| S1n | Fn | An |

In an embodiment, the original myoelectric signal S1 is decomposed by the decomposer 151 using a signal decomposition algorithm, which is a combination of a short time Fourier transform (STFT) and a power spectral density function (PSDF), a small wave transform algorithm, or an empirical mode decomposition (EMD) algorithm.

Afterwards, the method proceeds to step S152, the amplitude variations Ai of the myoelectric sub-signals S1i are respectively adjusted by the adjuster 152 according to the capacitance value CV of the skin 900. Referring to FIG. 5, a schematic diagram of adjusting a particular myoelectric sub-signal S1i is shown. The amplitude variation Ai of the myoelectric sub-signal S1i only has 24.43 mV. The adjuster 152 adjusts the amplitude variation Ai of the myoelectric sub-signal S1i to amplitude variation Ai* according to an adjustment ratio (such as 51.1%). The adjusted amplitude variation Ai* can be 50 mV. In comparison to the ideal myoelectric sub-signal S0i, the adjusted myoelectric sub-signal S1i* has an accuracy of 99.61%.

In an embodiment, for different myoelectric sub-signals S1i, the adjustment ratios for adjusting the amplitude variations Ai are not necessarily identical. The adjuster 152 can inquire corresponding adjustment ratio according to the capacitance value CV and the frequency Fi. The adjuster 152 can adjust all myoelectric sub-signals S1i to obtain complete adjusted myoelectric sub-signals S1i*.

Then, the method proceeds to step S153, the myoelectric sub-signals S1i* adjusted by the adjuster 152 are merged by the merger 153 using an anti-Fourier transform algorithm (frequency domain to time domain) to obtain a compensated myoelectric signal S1*.

As disclosed in above embodiments, after the original myoelectric signal S1 is obtained by the myoelectric signal sensing unit 110, the compensation unit 150 can compensate the original myoelectric signal S1 according to the capacitance value CV of the skin 900 obtained by the skin sensing unit 120 to obtain the compensated myoelectric signal S1*. The compensated myoelectric signal S1* resolves the problem of impedance mismatch, such that the coupled physiological signal measurement system 100 adopting low pressure sensing or non-contact sensing also can obtain a measurement result with high accuracy.

The step S120 and the skin sensing unit 120 disclosed above can be realized through different implementations which are disclosed below respectively.

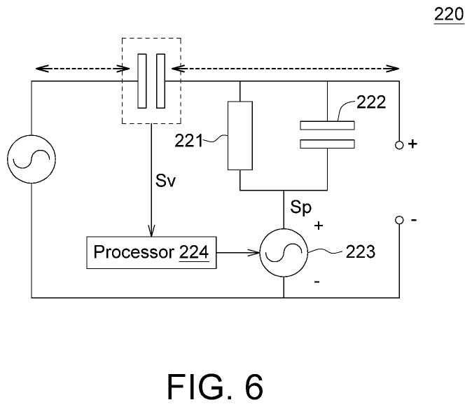

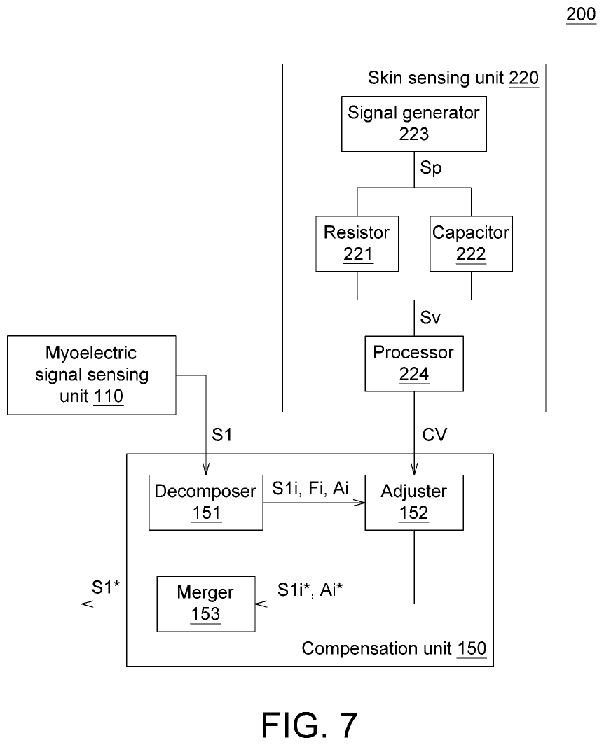

Referring to FIG. 6, a schematic diagram of a skin sensing unit 220 according to an embodiment is shown. The skin sensing unit 220 includes a resistor 221, a capacitor 222, a signal generator 223 and a processor 224. In the present embodiment, the resistor 221 and the capacitor 222 are connected in parallel. However, in another embodiment, the resistor 221 and the capacitor 222 can also be connected in series. The signal generator 223 inputs a square wave signal Sp to the parallel circuit (or series circuit) of the resistor 221 and the capacitor 222. After a voltage sensing signal Sv is obtained by the processor 224, the capacitance value CV of the skin 900 (illustrated in FIG. 7) can then be obtained by analyzing the voltage sensing signal Sv. Details of the operations of each of the elements disclosed above are disclosed below with a flowchart.

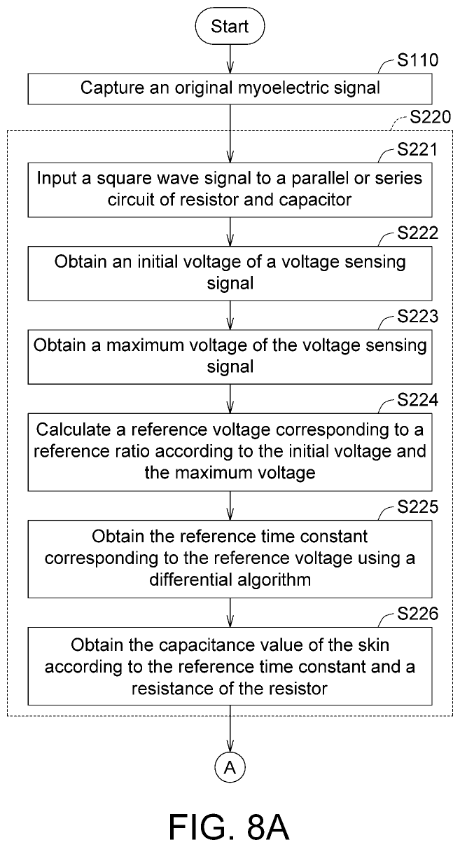

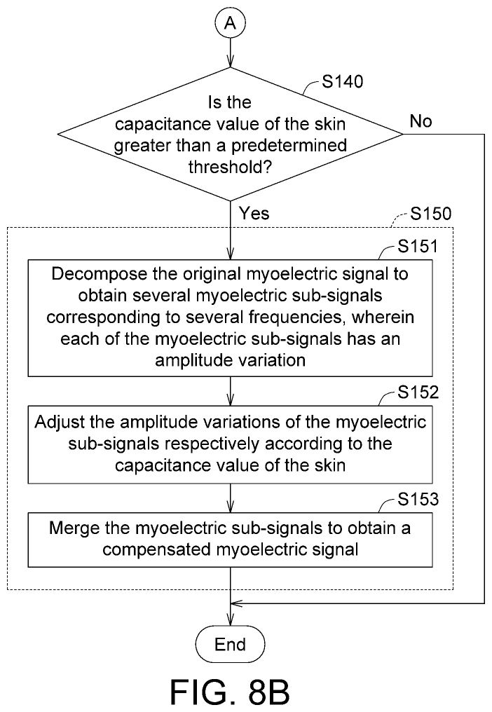

Referring to FIG. 7 and FIG. 8A to 8B. FIG. 7 is a block diagram of a coupled physiological signal measurement system 200 according to an embodiment. FIGS. 8A to 8B are flowcharts of a coupled physiological signal measurement method according to an embodiment. In step S220, a capacitance value CV of a skin 900 is obtained by the skin sensing unit 220. Step S220 includes steps S221 to S226.

In step S221, a square wave signal Sp is inputted to a parallel or series circuit of the resistor 221 and the capacitor 222 by the signal generator 223. The square wave signal Sp is a signal with periodic change, such as a pulse-width modulation (PWM) signal. After the square wave signal Sp is inputted to the parallel circuit (or series circuit) of the resistor 221 and the capacitor 222, the processor 224 can obtain the voltage sensing signal Sv.

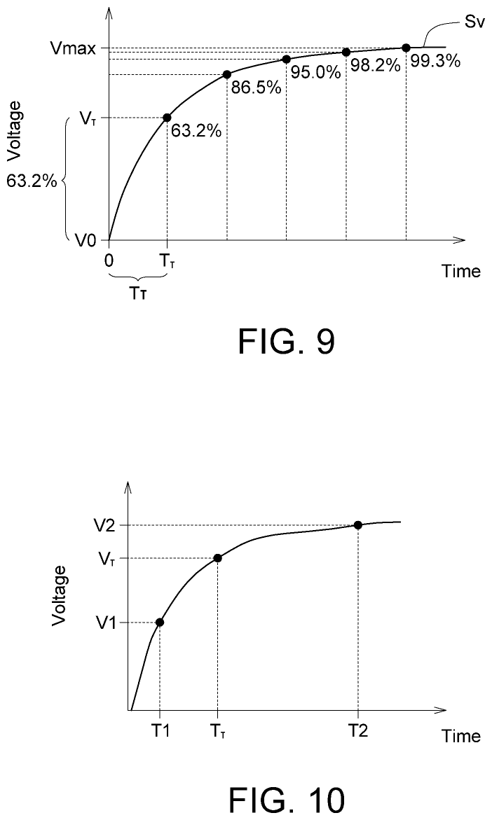

Referring to FIG. 9, a schematic diagram illustrating the voltage sensing signal Sv is shown. The voltage sensing signal Sv obtained by the processor 224 also varies with the square wave signal Sp (illustrated in FIG. 7). For example, the voltage level of the voltage sensing signal Sv raises to the maximum voltage Vmax from an initial voltage V0. Since the capacitance value CV of the skin 900 affects the rising speed of the voltage sensing signal Sv, the processor 224 can obtain the capacitance value CV of the skin 900 by analyzing the rising speed of the voltage sensing signal Sv.

Then, the method proceeds to step S222, the initial voltage V0 of the voltage sensing signal Sv is obtained by the processor 224.

Afterwards, the method proceeds to step S223, the maximum voltage Vmax of the voltage sensing signal Sv is obtained by the processor 224.

Then, the method proceeds to step S224, a reference voltage V_{τ} corresponding to a reference ratio (such as 63.2%) is calculated by the processor 224 according to the initial voltage V0 and the maximum voltage Vmax. For example, the processor 224 calculates the reference voltage V_{τ} according to formula (1):

V0+(Vmax−V0)*63.2%=V_{τ} (1)

Afterwards, the method proceeds to step S225, the reference time constant T_{τ} corresponding to the reference voltage V_{τ} is obtained by the processor 224 using a differential algorithm. Referring to FIG. 10, a schematic diagram illustrating how the reference time constant T_{τ} is obtained using a differential algorithm is shown. When the processor 224 records voltage V1 and voltage V2 at unit time points T1 and T2 respectively, the reference voltage V_{τ} is between the voltage V1 and the voltage V2, and the reference time constant T_{τ} is also between the unit time point T1 and the unit time point T2. When the unit time point T1 and the unit time point T2 are very close to each other, the reference time constant T_{τ} can be obtained using a differential algorithm according to formula (2):

$$\frac{V 2 - {V}_{τ}}{V 2 - V 1} = \frac{T 2 - {T}_{τ}}{T 2 - T 1} \qquad ( 2 )$$

Then, the method proceeds to step S226, the capacitance value CV of the skin 900 is obtained by the processor 224 according to the reference time constant T_{τ} and a resistance RV of the resistor 221. For example, the processor 224 obtains the capacitance value CV of the skin 900 according to formula (3):

T_{τ}=RV*CV (3)

Thus, the skin sensing unit 220 can smoothly obtain the capacitance value CV of the skin 900. Details of steps S140 to S150 are already disclosed above and are not repeated here.

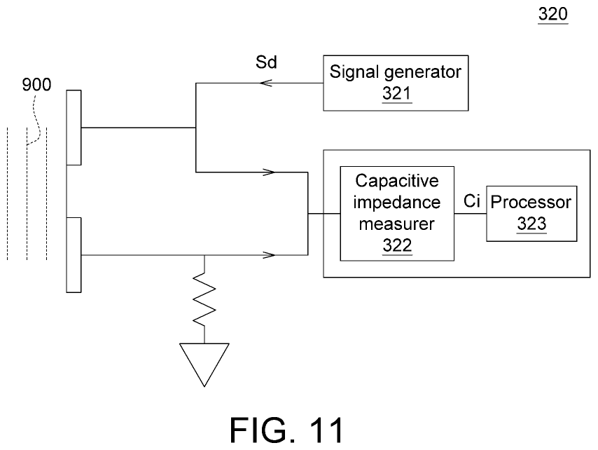

Apart from the above embodiments, the capacitance value CV of the skin 900 can be obtained through capacitive impedance. Referring to FIG. 11, a schematic diagram of a skin sensing unit 320 according to another embodiment is shown. The skin sensing unit 320 includes a signal generator 321, a capacitive impedance measurer 322 and a processor 323. The signal generator 321 is configured to input a DC signal Sd. The capacitive impedance measurer 322 obtains a capacitive impedance curve Ci corresponding to the DC signal Sd. Since the capacitive impedance curve Ci is affected by the capacitance value CV of the skin 900, the processor 323 can obtain the capacitance value CV of the skin 900 according to the capacitive impedance curve Ci. Details of the operations of each of the elements disclosed above are disclosed below with a flowchart.

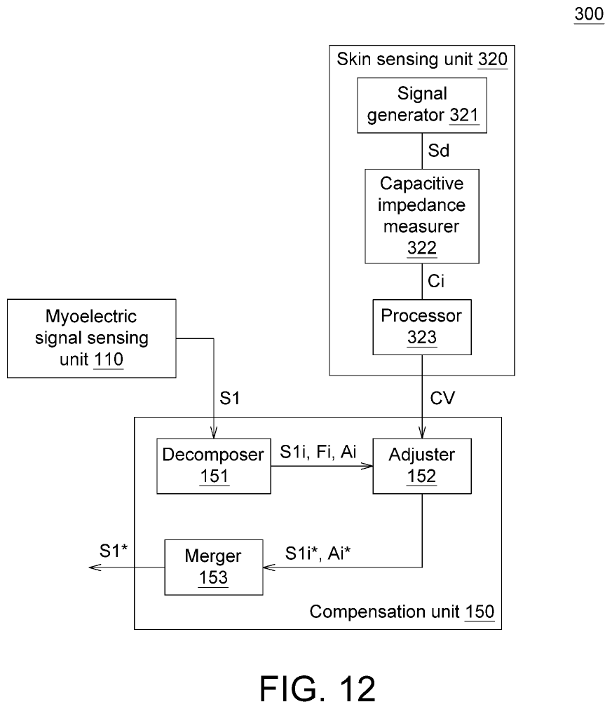

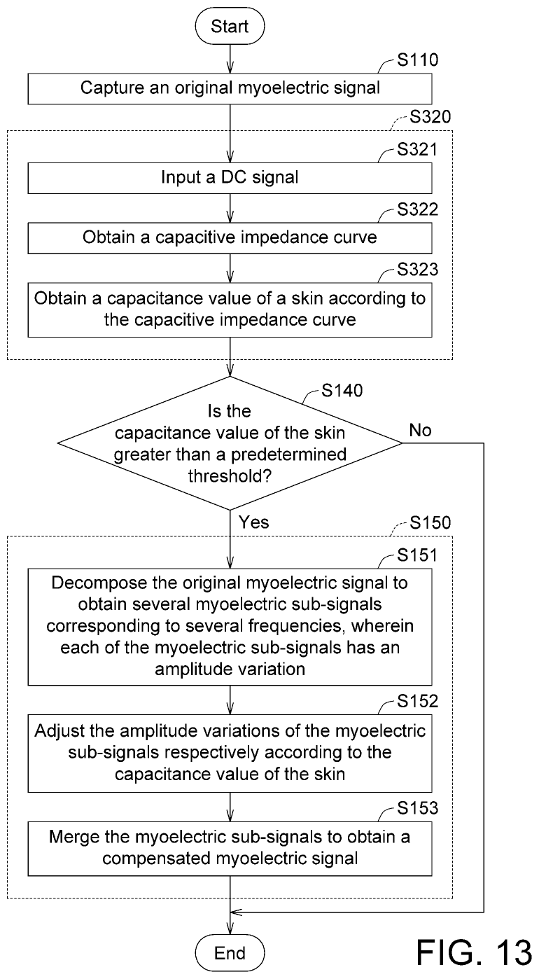

Referring to FIG. 12 and FIG. 13. FIG. 12 is a block diagram of a coupled physiological signal measurement system 300 according to an embodiment. FIG. 13 is a flowchart of a coupled physiological signal measurement method according to an embodiment. In step S320, the capacitance value CV of the skin 900 is obtained by the skin sensing unit 320. Step S320 includes steps S321 to S323.

In step S321, a DC signal Sd is inputted by the signal generator 321, wherein the voltage level of the DC signal Sd is pre-determined, and each time when the DC signal Sd is inputted, the voltage level of the DC signal Sd remains the same.

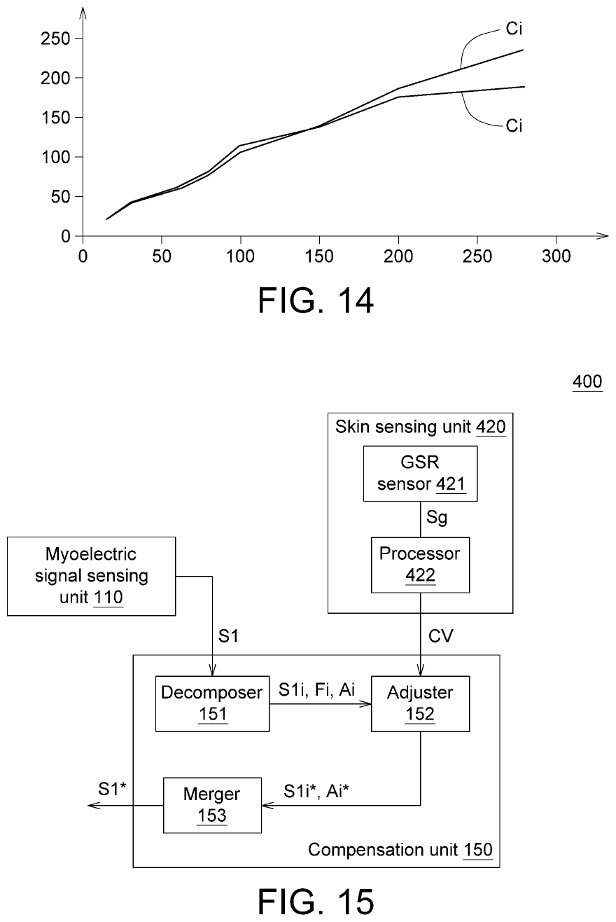

In step S322, a capacitive impedance curve Ci is obtained by the capacitive impedance measurer 322. Referring to FIG. 14, a schematic diagram illustrating various capacitive impedance curves Ci is shown. The capacitance value CV of the skin 900 affects the capacitive impedance curve Ci. Therefore, the capacitive impedance curve Ci can be recorded and used to analyze the corresponding capacitance value CV.

In step S323, a capacitance value CV of a skin 900 is obtained by the processor 323 according to the capacitive impedance curve Ci. For example, the processor 323 can analyze the capacitance value CV of the skin 900 according to the slope, mean and variance of the capacitive impedance curve Ci. Or, the processor 323 can recognize the capacitance value CV of the skin 900 corresponding to the capacitive impedance curve Ci using a machine learning algorithm.

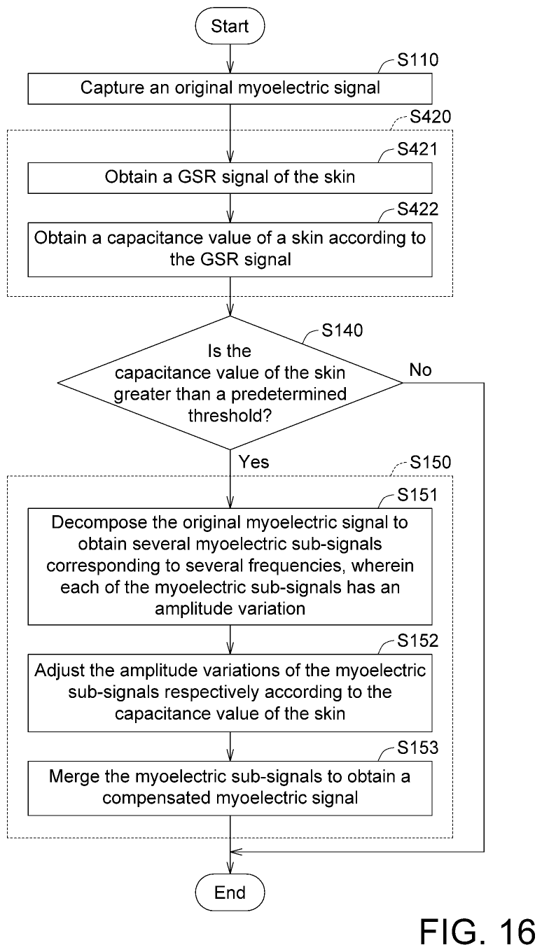

Apart from the above embodiments, the capacitance value CV of the skin 900 can further be obtained through the galvanic skin response (GSR) signal. Referring to FIG. 15 and FIG. 16. FIG. 15 is a block diagram of a coupled physiological signal measurement system 400 according to an embodiment. FIG. 16 is a flowchart of a coupled physiological signal measurement method according to an embodiment. As indicated in FIG. 15, the skin sensing unit 420 includes a GSR sensor 421 and a processor 422. In step S420, the capacitance value CV of the skin 900 is obtained by the skin sensing unit 420. Step S420 includes steps S421 to S422. In step S421, a GSR signal Sg of the skin 900 is obtained by the GSR sensor 421. The GSR sensor 421 can provide information related to the activities of sweat gland. The sweat gland and the sympathetic nerve are activated under excitation and pressure, and this information is referred as GSR signal Sg. The GSR signal Sg is found to closely affect the capacitance value CV of the skin 900.

In step S422, the capacitance value CV of the skin 900 is obtained by the processor 422 according to the GSR signal Sg. For example, the processor 422 can analyze the capacitance value CV of the skin 900 according to the mean and variance of the GSR signal Sg. Or, the processor 422 can recognize the capacitance value CV of the skin 900 corresponding to the GSR signal Sg using a machine learning algorithm.

According to the embodiments disclosed above, the capacitance value CV of the skin 900 can be accurately analyzed and further compensate the original myoelectric signal S1 according to the capacitance value CV of the skin 900 to obtain a compensated myoelectric signal S1*.

In some embodiments, each of the coupled physiological signal measurement systems 100, 200, 300 and 400 can be disposed on the functional clothing or the surface cloth of backpack and can have an indirect contact with the skin 900 through a layer of nylon cloth or a cotton cloth. To increase measurement accuracy, the interference caused by the capacitance value of the fabric 800 has been resolved in the said embodiments.

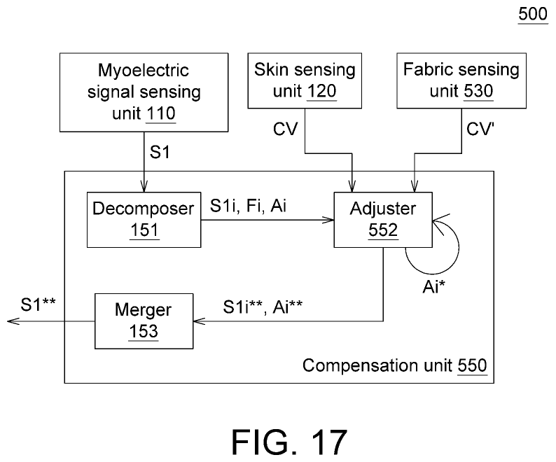

Referring to FIG. 17, a block diagram of a coupled physiological signal measurement system 500 according to an embodiment is shown. In the present embodiment, the coupled physiological signal measurement system 500 further includes a fabric sensing unit 530. The fabric sensing unit 530 is configured to obtain a capacitance value CV of a fabric 800. The capacitance value CV of the fabric 800 can be used to more accurately compensate the original myoelectric signal S1. The fabric sensing unit 530 can be realized by a circuit, a chip or a circuit board. Details of the operations of each of the elements disclosed above are disclosed below with a flowchart.

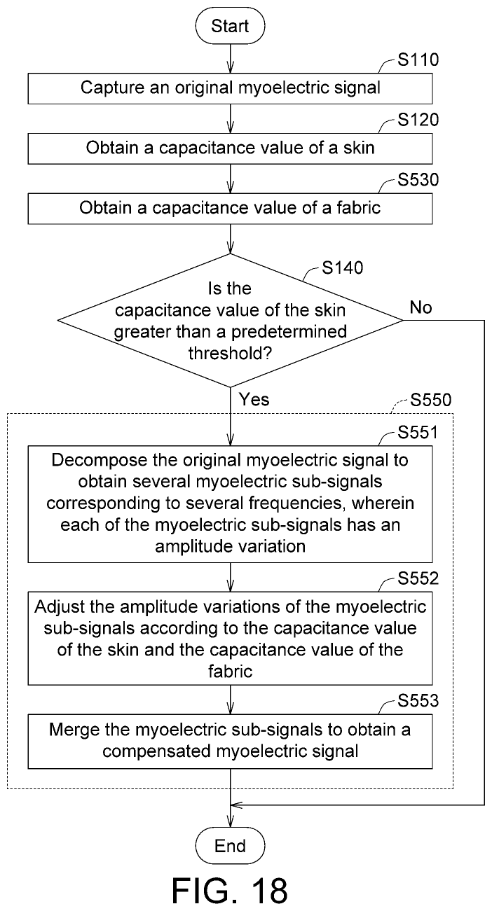

Referring to FIG. 18, a flowchart of a coupled physiological signal measurement method according to an embodiment is shown. In step S120, the capacitance value CV of the skin 900 is obtained, and the method proceeds to step S530.

In step S530, a capacitance value CV of a fabric 800 is obtained by the fabric sensing unit 530. The skin sensing unit 120 and the fabric sensing unit 530 are independent of each other and do not interfere with each other. Both the capacitance value CV of the skin 900 obtained by the skin sensing unit 120 and the capacitance value CV of the fabric 800 obtained by the fabric sensing unit 530 can be used to compensate the original myoelectric signal S1 to obtain an accurate compensated myoelectric signal S1*.

Then, the method proceeds to step S140, whether the capacitance value CV of the skin 900 is greater than a predetermined threshold is determined by the compensation unit 550. If the capacitance value CV is greater than the predetermined threshold, the method proceeds to step S550; if the capacitance value CV is not greater than the predetermined threshold, the method terminates. The predetermined threshold is set according to the user's age, weight, gender, height or medical history. Following compensation is activated only when the capacitance value CV is greater than the predetermined threshold. In the present step, the compensation unit 550 mainly determines the capacitance value CV of the skin 900 but not the capacitance value CV′ of the fabric 800.

Afterwards, the method proceeds to step S550, the original myoelectric signal S1 is compensated by the compensation unit 550 according to the capacitance value CV of the skin 900 and the capacitance value CV of the fabric 800. Step S550 includes step S551 to S553.

In step S551, the original myoelectric signal S1 is decomposed by the decomposer 151 to obtain several myoelectric sub-signals S1i corresponding to several frequencies Fi, wherein each myoelectric sub-signal S1i has an amplitude variation Ai. The amplitude variation Ai is such as a difference between an amplitude crest and an amplitude trough.

Afterwards, the method proceeds to step S552, the amplitude variations Ai of the myoelectric sub-signals S1i are adjusted by the adjuster 552 according to the capacitance value CV of the skin 900 and the capacitance value CV of the fabric 800. For example, the adjuster 552 adjusts the amplitude variation Ai of the myoelectric sub-signal S1i to an adjusted amplitude variation Ai* according to an adjustment ratio (such as 51.1%). Then, the adjuster 552 adjusts the amplitude variation Ai* to the amplitude variation Ai* according to an adjustment translation value. In response to the adjusted amplitude variation Ai**, the adjuster 552 outputs the adjusted myoelectric sub-signal S1i**.

In an embodiment, for different myoelectric sub-signals S1i, corresponding adjustment ratios and adjustment translation values are not necessarily identical. The adjuster 552 can inquire corresponding adjustment ratios and adjustment translation values according to the capacitance value CV and frequency Fi. The adjuster 152 can adjust all myoelectric sub-signals S1i to obtain a complete adjusted myoelectric sub-signal S1i*.

Then, the method proceeds to step S553, the adjusted myoelectric sub-signals S1i* is merged by the merger 153 to obtain a compensated myoelectric signal S1**.

According to the above embodiments, after the original myoelectric signal S1 is obtained by the myoelectric signal sensing unit 110, the compensation unit 550 can compensate the original myoelectric signal S1 to obtain the compensated myoelectric signal S1* according to the capacitance value CV of the skin 900 obtained by the skin sensing unit 12 and the capacitance value CV of the fabric 800 obtained by the fabric sensing unit 530. The compensated myoelectric signal S1* resolves the problem of impedance mismatch, such that the coupled physiological signal measurement system 100 adopting low pressure sensing or non-contact sensing also can obtain a measurement result with high accuracy.

In an embodiment, the myoelectric signal sensing unit 110, the skin sensing unit 120 and the fabric sensing unit 530 disclosed above can be integrated into a near-end device; the compensation units 150 and 550 can be disposed in a remote end device, such as a mobile phone, notebook computer or a server. Relevant operations can be displayed through a graphic user interface.

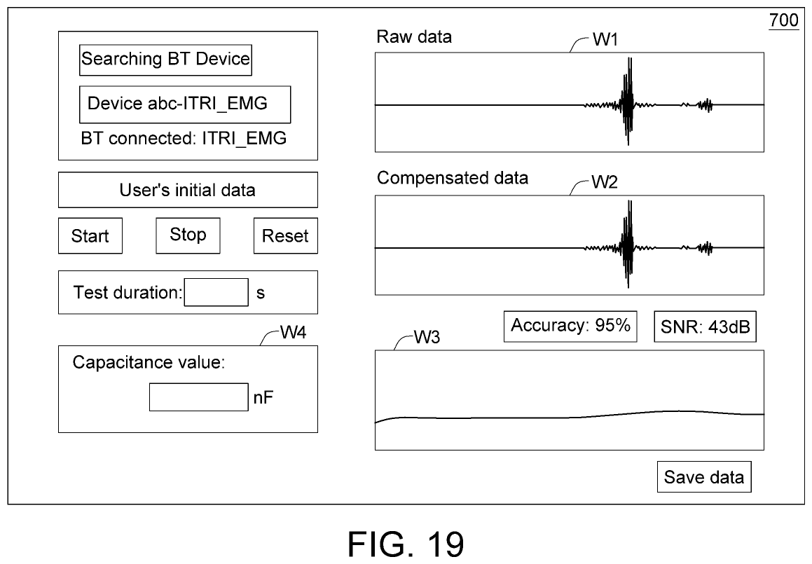

Referring to FIG. 19, a schematic diagram of a graphic user interface 700 according to an embodiment is shown. The graphic user interface 700 includes a first wave window W1, a second wave window W2, a third wave window W3 and a skin sensing information window W4. The first wave window W1 is configured to display the original myoelectric signal S1. The skin sensing information window W4 is configured to display the capacitance value CV of the skin 900. The second wave window W2 is configured to display the compensated myoelectric signal S1*, S1**. The third wave window W3 is configured to display the wave of the capacitance value CV of the skin 900.

According to the above embodiments, after the original myoelectric signal S1 is obtained, the original myoelectric signal S1 can be compensated according to the capacitance value CV of the skin 900 to obtain a compensated myoelectric signal S1* or can be compensated according to the capacitance value CV of the skin 900 and the capacitance value CV of the fabric 800 to obtain a compensated myoelectric signal S1**. The compensated myoelectric signals S1* and S1** can resolve the problem of impedance mismatch, such that the coupled physiological signal measurement systems 100 to 500 adopting low pressure sensing or non-contact sensing also can obtain a measurement result with high accuracy.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A coupled physiological signal measurement method, comprising:
capturing an original myoelectric signal;
obtaining a capacitance value of a skin; and
compensating the original myoelectric signal according to the capacitance value of the skin;
wherein the step of compensating the original myoelectric signal according to the capacitance value comprises:
decomposing the original myoelectric signal to obtain a plurality of myoelectric sub-signals corresponding to a plurality of frequencies, wherein each of the myoelectric sub-signals has an amplitude variation;
adjusting the amplitude variations of the myoelectric sub-signals respectively according to the capacitance value of the skin; and
merging the adjusted myoelectric sub-signals to obtain a compensated myoelectric signal.

2. The coupled physiological signal measurement method according to claim 1, wherein the step of obtaining the capacitance value of the skin comprises:
inputting a square wave signal to a parallel or series circuit of a resistor and a capacitor;
obtaining an initial voltage of a voltage sensing signal;
obtaining a maximum voltage of the voltage sensing signal;
calculating a reference voltage corresponding to a reference ratio according to the initial voltage and the maximum voltage;
obtaining a reference time constant corresponding to the reference voltage using a differential algorithm; and
obtaining the capacitance value of the skin according to the reference time constant and a resistance of the resistor.

3. The coupled physiological signal measurement method according to claim 1, wherein the step of obtaining the capacitance value of the skin comprises:
inputting a direct current (DC) signal;
obtaining a capacitive impedance curve; and
obtaining the capacitance value of the skin according to the capacitive impedance curve.

4. The coupled physiological signal measurement method according to claim 1, wherein the step of obtaining the capacitance value of the skin comprises:
obtaining a galvanic skin response (GSR) signal of the skin; and
obtaining the capacitance value of the skin according to the GSR signal.

5. The coupled physiological signal measurement method according to claim 1, wherein the original myoelectric signal is decomposed using a signal decomposition algorithm, which is a combination of a short time Fourier transform (STFT) and a power spectral density function (PSDF), or a small wave transform algorithm, or an empirical mode decomposition (EMD) algorithm.

6. The coupled physiological signal measurement method according to claim 1, wherein the amplitude variation of each of the myoelectric sub-signals is compensated according to an adjustment ratio.

7. The coupled physiological signal measurement method according to claim 6, wherein the adjustment ratios of the amplitude variations are not identical.

8. The coupled physiological signal measurement method according to claim 1, further comprising:
obtaining a capacitance value of a fabric; and
compensating the original myoelectric signal according to the capacitance value of the fabric.

9. The coupled physiological signal measurement method according to claim 1, wherein the original myoelectric signal is compensated only when the capacitance value of the skin is greater than a predetermined threshold.

10. A coupled physiological signal measurement system, comprising:
a myoelectric signal sensing unit, configured to capture an original myoelectric signal;
a skin sensing unit, configured to obtain a capacitance value of a skin; and
a compensation unit, configured to compensate the original myoelectric signal according to the capacitance value of the skin;
wherein the compensation unit comprises:
a decomposer, configured to decompose the original myoelectric signal to obtain a plurality of myoelectric sub-signals corresponding to a plurality of frequencies, wherein each of the myoelectric sub-signals has an amplitude variation;
an adjuster, configured to adjust the amplitude variations of the myoelectric sub-signals respectively according to the capacitance value of the skin; and
a merger, configured to merge the adjusted myoelectric sub-signals to obtain a compensated myoelectric signal.

11. The coupled physiological signal measurement system according to claim 10, wherein the skin sensing unit comprises:
a resistor;
a capacitor, wherein the resistor and the capacitor are connected in parallel or series;
a signal generator, configured to input a square wave signal to a parallel or series circuit of the resistor and the capacitor; and
a processor, configured to obtain an initial voltage of a voltage sensing signal and a maximum voltage and to calculate a reference voltage corresponding to a reference ratio according to the initial voltage and the maximum voltage, wherein the processing unit obtains a reference time constant corresponding to the reference voltage using a differential algorithm, and further obtains the capacitance value of the skin according to the reference time constant and a resistance of the resistor.

12. The coupled physiological signal measurement system according to claim 10, wherein the skin sensing unit comprises:
a signal generator, configured to input a DC signal;
a capacitive impedance measurer, configured to obtain a capacitive impedance curve; and
a processor, configured to obtain the capacitance value of the skin according to the capacitive impedance curve.

13. The coupled physiological signal measurement system according to claim 10, wherein the skin sensing unit comprises:
a galvanic skin response (GSR) sensor, configured to obtain a GSR signal of the skin; and
a processor, configured to obtain the capacitance value of the skin according to the GSR signal.

14. The coupled physiological signal measurement system according to claim 10, wherein the decomposer decomposes the original myoelectric signal using a signal decomposition algorithm, which is a combination of a short time Fourier transform (STFT) and a power spectral density function (PSDF), or a small wave transform algorithm, or an empirical mode decomposition (EMD) algorithm.

15. The coupled physiological signal measurement system according to claim 10, wherein the adjuster adjusts the amplitude variation of each of the myoelectric sub-signals according to an adjustment ratio.

16. The coupled physiological signal measurement system according to claim 15, wherein the adjustment ratios of the amplitude variations are not identical.

17. The coupled physiological signal measurement system according to claim 10, further comprising:
a fabric sensing unit, configured to obtain a capacitance value of a fabric;
wherein the compensation unit further compensates the original myoelectric signal according to the capacitance value of the fabric.

18. The coupled physiological signal measurement system according to claim 10, wherein the compensation unit compensates the original myoelectric signal only when the capacitance value of the skin is greater than a predetermined threshold.

19. A graphic user interface, comprising:
a first wave window, configured to display an original myoelectric signal;
a skin sensing information window, configured to display a capacitance value of a skin; and
a second wave window, configured to display a compensated myoelectric signal, wherein the original myoelectric signal is adjusted according to the capacitance value of the skin to obtain the compensated myoelectric signal.

20. The graphic user interface according to claim 19, further comprising:
a third wave window, configured to display a wave of the capacitance value of the skin.
